(19) United States
(12) Patent Application Publication
(10) Pub. No.: US 2022/0040382 A1
(43) Pub. Date: Feb. 10, 2022

(54) HYDROGEL CONTAINING POLYMER NANOFIBER HAVING SULFATE GROUP INTRODUCED AND METHOD FOR PREPARING THE SAME

(71) Applicant: GWANGJU INSTITUTE OF SCIENCE AND TECHNOLOGY, Gwangju (KR)

(72) Inventors: Sungrok Wang, Gwangju (KR); Myung-Han YOON, Gwangju (KR)

(21) Appl. No.: 17/395,956

(22) Filed: Aug. 6, 2021

(30) Foreign Application Priority Data
Aug. 6, 2020 (KR) 10-2020-0098581

(51) Int. Cl. A61L 27/52; A61L 27/48; B33Y 80/00; B33Y 70/10

(52) U.S. Cl. CPC A61L 27/52; A61L 27/48; B82Y 5/00; B33Y 70/10; A61L 2400/12; B33Y 80/00

(57) ABSTRACT

Proposed are a hydrogel containing a polymer nanofiber into which a sulfate group is introduced, and a method for preparing the same. The hydrogel contains polymer compound nanofibers into which a sulfate group is introduced, the polymer compound nanofibers may be connected to each other by a crosslink to form a network structure, and gelling may proceed as water is filled in empty space of the network structure. According to an embodiment, it is possible to provide a hydrogel containing a polymer nanofiber into which a sulfate group is introduced, which is highly utilizable as an artificial extracellular matrix by providing a cell culture scaffold which exhibits high cell adhesiveness and of which the three-dimensional structure can be controlled.

### BACKGROUND OF THE INVENTION

### Field of the Invention

The present invention relates to a hydrogel prepared using a polymer nanofiber into which a sulfate group is introduced. More specifically, the present invention relates to a hydrogel containing a polymer nanofiber into which a sulfate group is introduced, which can be utilized as a biomaterial or artificial extracellular matrix.

### Description of the Related Art

Animal tissues are generally composed of cells and extracellular matrix (ECM). In particular, the extracellular matrix contains polysaccharides belonging to glycosaminoglycans (GAG) and structural proteins such as collagen, and this allows the tissues to form a three-dimensional shape.

Studies have been actively conducted to utilize such animal tissues in tissue engineering by culturing cells in an in vitro environment and thus producing artificial organs and artificial tissues. However, most animal cells exhibit anchorage dependence and density dependence and thus cannot form a specific structure by itself in an in-vitro environment without special conditions. Hence, in order to culture animal cells into a three-dimensional structure in an in-vitro environment, it is significantly important to form an appropriate scaffold that serves as an extracellular matrix.

In the prior art, alginate, GelMA and the like have been used as a scaffold, but there have been problems that the scaffold exhibits poor cell adhesiveness, or is simply in the form of a thin film, or does not have a shape so that the three-dimensional structure of the scaffold cannot be easily controlled.

### CITATION LIST

### Patent Literature

Patent Literature 1: Korea Patent No. 10-1102308

### SUMMARY OF THE INVENTION

A technical object to be achieved by the present invention is to provide a hydrogel having a network structure, of which the degree of crosslinking and the degree of water swelling can be adjusted and the three-dimensional structure can be finely controlled by the crosslinking between polymer compound nanofibers and the introduction of a sulfate group.

A technical object to be achieved by the present invention is to provide a hydrogel which is highly utilizable for fine control of a three-dimensional structure and as an artificial extracellular matrix through a configuration that precisely mimics the extracellular matrix in vivo.

The technical objects to be achieved by the present invention are not limited to the technical objects mentioned above, and other technical objects not mentioned will be clearly understood by those skilled in the art to which the present invention pertains from the following description.

In order to achieve the technical objects, an embodiment of the present invention provides a hydrogel containing a polymer nanofiber into which a sulfate group is introduced, which comprises polymer compound nanofibers into which a sulfate group is introduced and in which the polymer compound nanofibers are connected to each other by a crosslink to form a network structure and gelling proceeds as water is filled in empty space of the network structure.

The polymer compound may include at least any one selected from the group consisting of collagen, gelatin, elastin, fibrin, and fibronectin.

In order to achieve the technical objects, another embodiment of the present invention provides a method for preparing a hydrogel containing a polymer nanofiber into which a sulfate group is introduced, which comprises: a polymer compound solution preparing step; a crosslinking agent adding step of adding a crosslinking agent to the solution; an electrospinning step of electrospinning the solution to form the polymer compound into polymer compound nanofibers; a crosslink forming step of forming a crosslink between the polymer compound nanofibers; a sulfate group introducing step of introducing a sulfate group into the polymer compound nanofibers; and a hydrogel obtaining step of hydrating the resultant into which a sulfate group has been introduced to obtain a hydrogel.

The polymer compound may include at least any one selected from the group consisting of collagen, gelatin, elastin, fibrin, and fibronectin.

The diameter of the polymer compound nanofibers may be controlled by adjusting an electrospinning condition in the electrospinning step.

The crosslinking agent may contain a reducing sugar selected from the group consisting of glucose, fructose, maltose, lactose, and galactose, an organic acid having two or more carboxyl groups selected from the group consisting of sebacic acid, citric acid, and tartaric acid, and at least any one selected from the group consisting of glutaraldehyde, genipin, and carbodiimide.

The crosslinking agent may be added at a weight ratio of 0.06 to 0.2 with respect to the polymer compound.

In the sulfate group introducing step, a sulfate group may be introduced into the polymer compound nanofibers by treating the polymer compound nanofibers with a sulfating agent.

The sulfating agent may include at least any one selected from the group consisting of concentrated sulfuric acid, chlorosulfonic acid, sulfur trioxide, dimethylformamide-sulfur trioxide complex, pyridine-sulfur trioxide complex, and oleum.

### BRIEF DESCRIPTION OF THE DRAWINGS

FIG. 1 is a schematic diagram illustrating a hydrogel containing a polymer nanofiber into which a sulfate group is introduced according to an embodiment of the present invention;

FIG. 2 is a flowchart schematically illustrating a method for preparing a hydrogel containing a polymer nanofiber into which a sulfate group is introduced according to an embodiment of the present invention;

FIG. 3A is a graph illustrating the diameters of gelatin nanofibers in hydrogels prepared according to Preparation Examples 2 to 4;

FIG. 3B is a photograph of the thicknesses of gelatin nanofibers in hydrogels prepared according to Preparation Examples 2 to 4 taken using a scanning electron microscope;

FIGS. 3C, 3D, and 3E are graphs illustrating the diameters of gelatin nanofibers in hydrogels prepared according to Preparation Examples 5 to 7, Preparation Examples 8 to 10, and Preparation Examples 11 to 13, respectively;

FIG. 4A is a photograph of a hydrogel prepared according to Comparative Example 1 and a photograph thereof taken using a confocal laser scanning microscope; and

FIG. 4B is a photograph of a hydrogel prepared according to Preparation Example 1 and a photograph thereof taken using a confocal laser scanning microscope.

### DETAILED DESCRIPTION OF THE PREFERRED EMBODIMENTS

Hereinafter, the present invention will be described with reference to the accompanying drawings. However, the present invention may be embodied in several different forms, and thus is not limited to the embodiments described herein. In order to clearly explain the present invention in the drawings, parts irrelevant to the description are omitted, and similar reference numerals are attached to similar parts throughout the specification.

Throughout the specification, when a part is said to be “connected (linked, in contact, coupled)” with another part, this includes not only a case of being “directly connected” but also a case of being “indirectly connected” with another member interposed therebetween. In addition, when a part “includes” a certain component, this means that other components may be further provided rather than excluding other components unless otherwise stated.

The terminology used herein is used only to describe specific embodiments, and is not intended to limit the present invention. The singular expression includes the plural expression unless the context clearly dictates otherwise. In this specification, it should be understood that terms such as “include” or “have” are intended to designate that the features, numbers, steps, operations, components, parts, or combinations thereof described in the specification exist but do not preclude in advance the possibility of the presence or addition of one or more other features, numbers, steps, operations, components, parts, or combinations thereof.

Hereinafter, embodiments of the present invention will be described in detail with reference to the accompanying drawings.

A hydrogel containing polymer nanofibers into which sulfate groups are introduced according to an embodiment of the present invention will be described.

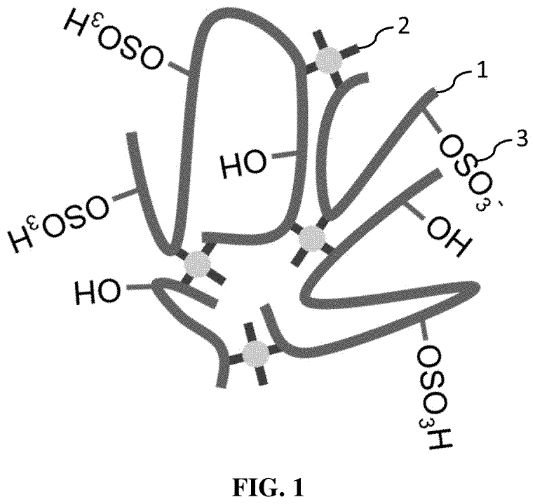

FIG. 1 is a schematic diagram illustrating the hydrogel containing polymer nanofibers into which sulfate groups are introduced according to an embodiment of the present invention.

Referring to FIG. 1, the hydrogel containing polymer nanofibers into which sulfate groups are introduced according to an embodiment of the present invention contains polymer compound nanofibers (1) into which sulfate groups (3) are introduced, the polymer compound nanofibers (1) are connected to each other by a crosslink (2) to form a network structure, and gelling proceeds as water is filled in the empty space of the network structure.

For example, the polymer compound may include at least any one selected from the group consisting of collagen, gelatin, elastin, fibrin, and fibronectin. It may be preferable to use a natural polymer which exhibits high cell adhesiveness and is easily biodegraded, and it may be more preferable to use gelatin that has three or more kinds of functional groups to allow a significantly wide range of crosslinking agents which can be used for crosslinking, is a mixture of hydrolyzed collagen chains, thus preserves a part of the complicated protein structure (helix and the like) that collagen previously had, and is thus easily formed into a three-dimensional shape.

Because of the configuration, according to an embodiment of the present invention, it is possible to provide a hydrogel containing polymer nanofibers into which sulfate groups are introduced, which exhibits excellent cell adhesiveness, is easily biodegraded, is easily formed into a three-dimensional structure, and is thus highly utilizable as a tissue engineering biomaterial.

Because of the configuration, according to an embodiment of the present invention, it is also possible to provide a hydrogel containing polymer nanofibers into which sulfate groups are introduced, which is highly utilizable as a biomaterial such as a three-dimensional cell culture scaffold and a bio-ink for 3D bioprinting technology.

Because of the configuration, according to an embodiment of the present invention, it is also possible to provide a hydrogel containing polymer nanofibers into which sulfate groups are introduced, which is highly utilizable as an artificial extracellular matrix through a configuration that precisely mimics the extracellular matrix in vivo.

The method for preparing a hydrogel containing polymer nanofibers into which sulfate groups are introduced according to another embodiment of the present invention will be described.

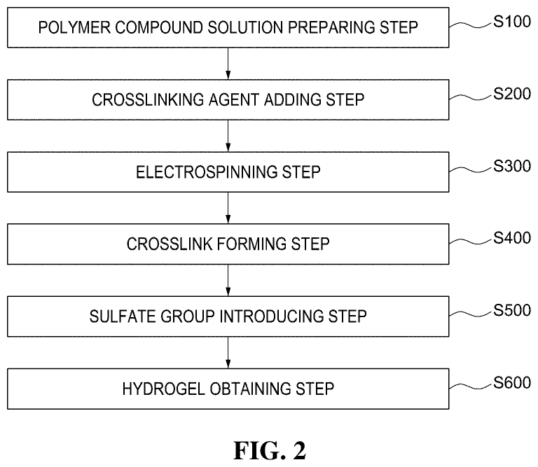

FIG. 2 is a flowchart schematically illustrating the method for preparing a hydrogel containing polymer nanofibers into which sulfate groups are introduced according to an embodiment of the present invention.

Referring to FIG. 2, the method for preparing a hydrogel containing polymer nanofibers into which sulfate groups are introduced according to an embodiment of the present invention may include a polymer compound solution preparing step (S100); a crosslinking agent adding step (S200) of adding a crosslinking agent to the solution; an electrospinning step (S300) of electrospinning the solution to form the polymer compound into polymer compound nanofibers; a crosslink forming step (S400) of forming a crosslink between the polymer compound nanofibers; a sulfate group introducing step (S500) of introducing a sulfate group into the polymer compound nanofibers; and a hydrogel obtaining step (S600) of hydrating the resultant into which a sulfate group has been introduced to obtain a hydrogel.

In the polymer compound solution preparing step (S100), the polymer compound solution may be prepared by dissolving the polymer compound in any one solvent selected from the group consisting of water and acetic acid.

The polymer compound may include at least any one selected from the group consisting of collagen, gelatin, elastin, fibrin, and fibronectin. It may be preferable to use a natural polymer which exhibits high cell adhesiveness and is easily biodegraded, and it may be more preferable to use gelatin that has three or more kinds of functional groups to allow a significantly wide range of crosslinking agents that can be used for crosslinking, is a mixture of hydrolyzed collagen chains, thus preserves a part of the complicated protein structure (helix and the like) that collagen previously had, and is thus easily formed into a three-dimensional shape.

The crosslinking agent to be added in the crosslinking agent adding step (S200) may contain a reducing sugar selected from the group consisting of glucose, fructose, maltose, lactose, and galactose, an organic acid having two or more carboxyl groups selected from the group consisting of sebacic acid, citric acid, and tartaric acid, and at least any one selected from the group consisting of glutaraldehyde, genipin, and carbodiimide.

It may be most preferable to use citric acid that is a natural crosslinking agent, has a small molecular size, is non-toxic, and is thus harmless to the human body.

For example, the crosslinking agent may be added at a weight ratio of 0.06 to 0.2 with respect to the polymer compound.

For example, the concentration of the crosslinking agent in a 15 w/v % gelatin solution may be from 1 w/v % to 3 w/v %.

In the electrospinning step (S300), the polymer compound may be formed into a nanofiber by electrospinning the polymer compound dissolved in a solution state.

When the polymer compound is directly gelled without the process of electrospinning the polymer compound to be formed into nanofibers, the hydrogel obtained in this way only forms a thin film composed of one layer but does not have a three-dimensional structure and is thus not suitable to be utilized as a three-dimensional cell culture scaffold.

Hence, by performing the process of forming the polymer compound into nanofibers in the electrospinning step (S300) and then crosslinking the nanofibers to form a network structure in which the polymer compound chains are entangled with each other, it is possible to provide a hydrogel which can be easily formed into a three-dimensional structure.

In the electrospinning step (S300), the diameter of the polymer compound nanofibers may be controlled by adjusting the electrospinning condition.

In the crosslink forming step (S400), crosslink forming methods including heat treatment, aldehyde vapor treatment, and ultraviolet light treatment may be used to form crosslinks between the electrospun polymer compound nanofibers.

In the sulfate group introducing step (S500), a sulfuric acid group may be introduced into the polymer compound by treating the polymer compound nanofibers with a sulfating agent.

For example, the sulfating agent may include at least any one selected from the group consisting of concentrated sulfuric acid, chlorosulfonic acid, sulfur trioxide, dimethylformamide-sulfur trioxide complex, pyridine-sulfur trioxide complex, and oleum.

Animal tissues are generally composed of cells and extracellular matrix (ECM). In particular, the extracellular matrix contains polysaccharides belonging to glycosaminoglycans (GAG) and structural proteins such as collagen, and this allows the tissues to form a three-dimensional shape.

Here, a glycosaminoglycan (GAG) is a long, branchless polysaccharide consisting of a repeated structure of a disaccharide to which a sulfate group is added, can interact with materials present in a living body such as growth factors, hormones, and cytokines in the extracellular matrix, and strongly interacts with integrin on the cell surface to help promote cell adhesion or growth.

It has been attempted to use GAG directly obtained from nature in order to create an environment similar to that in vivo when cells are cultured in an in vitro environment, but the unit cost thereof is high, and thus a sulfate group has been introduced into polysaccharides derived from seaweed or polysaccharides (for example, fucoidan) having a sulfate group have been used as artificial substitutes for GAG.

However, the present invention includes a configuration in which a sulfate group is directly introduced into a structural protein rather than a polysaccharide so that the structural protein can substitute for GAG.

Because of the configuration, according to an embodiment of the present invention, it is possible to provide a hydrogel containing polymer nanofibers into which sulfate groups are introduced, of which not only the three-dimensional structure is easily formed but also the microstructure can be controlled by adjusting the diameter of the nanofibers.

Because of the configuration, according to an embodiment of the present invention, it is also possible to provide a hydrogel containing polymer nanofibers into which sulfate groups are introduced, of which the degree of swelling after hydration and mechanical properties can be adjusted by adjusting the degree of crosslinking.

Because of the configuration, according to an embodiment of the present invention, it is also possible to provide a hydrogel containing polymer nanofibers into which sulfate groups are introduced, which can be utilized as a cell culture scaffold that closely mimics not only the appearance of the extracellular matrix in vivo but also the chemical properties thereof.

A three-dimensional artificial cell culture scaffold according to still another embodiment of the present invention will be described.

The three-dimensional artificial cell culture scaffold contains the hydrogel containing polymer nanofibers into which sulfate groups are introduced according to an embodiment of the present invention.

A bio-ink for 3D printing according to yet another embodiment of the present invention will be described.

The bio-ink for 3D printing contains the hydrogel containing polymer nanofibers into which sulfate groups are introduced according to an embodiment of the present invention.

Hereinafter, the present invention will be described in more detail with reference to Preparation Examples, Comparative Examples, and Experimental Examples. However, the present invention is not limited to the following Preparation Examples and Experimental Examples.

### Preparation Example 1: Preparation of Hydrogel Containing Polymer Nanofiber into Which Sulfate Group is Introduced

A gelatin solution was prepared by dissolving gelatin as a polymer compound in acetic acid. Citric acid to serve as a crosslinking agent was added to the gelatin solution. Gelatin nanofibers were obtained by electrospinning the gelatin solution containing citric acid. The gelatin nanofibers were heated to form crosslinks. Pyridine-sulfur trioxide complex (pyridine-SO₃ complex) as a sulfating agent was dissolved in dimethylformamide, and then the gelatin nanofibers thus crosslinked were treated with this solution to introduce sulfate groups into the gelatin nanofibers. The gelatin nanofibers into which sulfate groups had been introduced were hydrated to obtain a hydrogel.

### Preparation Examples 2 to 4

Hydrogels were prepared in the same manner as in Preparation Example 1 except that the percent concentration (w/v %) of gelatin in the solution was changed. At this time, in Preparation Examples 2 to 4, hydrogels were prepared so that the percent concentration (w/v %) of gelatin in the solution was 12 w/v %, 15 w/v %, and 18 w/v %, respectively.

### Preparation Examples 5 to 7

Hydrogels were prepared in the same manner as in Preparation Example 1 except that the distance between electrodes during the electrospinning was changed. At this time, in Preparation Examples 5 to 7, hydrogels were prepared so that the distances between electrodes during electrospinning was 13 cm, 16 cm, and 19 cm, respectively.

### Preparation Examples 8 to 10

Hydrogels were prepared in the same manner as in Preparation Example 1 except that the jetting speed of the solution during the electrospinning was changed. At this time, in Preparation Examples 8 to 10, hydrogels were prepared so that the solution jetting speed during electrospinning was 1.3 ml/h, 2.3 ml/h, and 4.3 ml/h, respectively.

### Preparation Examples 11 to 13

Hydrogels were prepared in the same manner as in Preparation Example 1 except that the concentration of citric acid (used as a crosslinking agent) in the solution was changed. At this time, in Preparation Examples 11 to 13, hydrogels were prepared so that the citric acid concentration in the solution was 0 w/v %, 1 w/v %, and 2 w/v %, respectively.

### Comparative Example 1

A hydrogel was prepared in the same manner as in Preparation Example 1 except that a sulfate group was not introduced into the gelatin nanofibers.

### Experimental Example 1

An experiment was conducted to measure changes in the diameter of the gelatin nanofibers during electrospinning under each condition. To this end, the gelatin nanofibers in the hydrogels prepared according to Preparation Examples 2 to 13 were observed under a scanning electron microscope and the diameters thereof were measured.

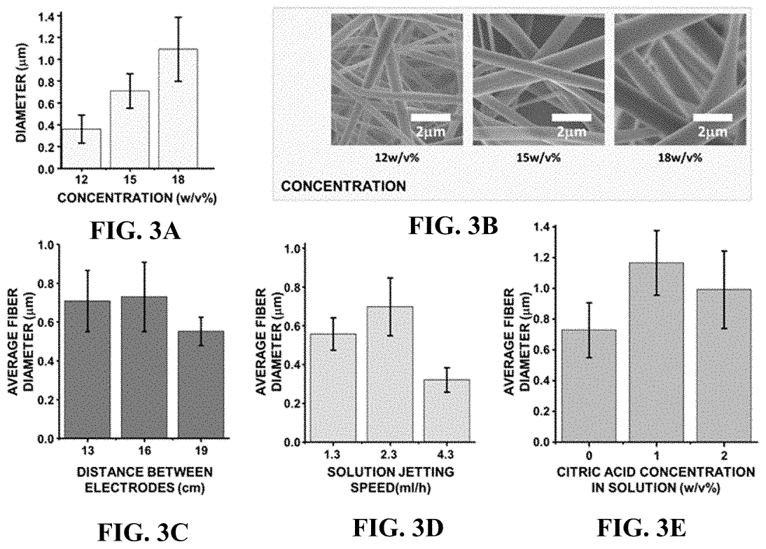

FIG. 3A is a graph illustrating the diameters of the gelatin nanofibers in the hydrogels prepared according to Preparation Examples 2 to 4.

FIG. 3B is a photograph of the thicknesses of the gelatin nanofibers in the hydrogels prepared according to Preparation Examples 2 to 4 taken using a scanning electron microscope.

FIGS. 3C, 3D, and are graphs illustrating the diameters of the gelatin nanofibers in the hydrogels prepared according to Preparation Examples 5 to 7, Preparation Examples 8 to 10, and Preparation Examples 11 to 13, respectively.

Referring to FIG. 3, it can be seen that the diameter of the polymer compound nanofibers can also be adjusted when the electrospinning condition is changed in the electrospinning step (S200).

### Experimental Example 2

An experiment was conducted to examine changes in the physical properties and degree of water swelling of a hydrogel by the introduction of a sulfate group.

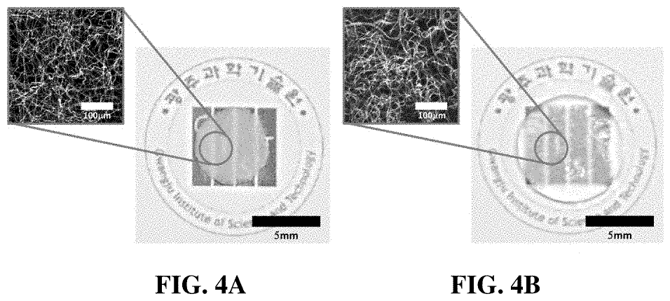

FIG. 4A is a photograph of the hydrogel prepared according to Comparative Example 1 and a photograph thereof taken using a confocal laser scanning microscope.

FIG. 4B is a photograph of the hydrogel prepared according to Preparation Example 1 and a photograph thereof taken using a confocal laser scanning microscope.

Referring to FIG. 4, it can be seen that when a sulfate group is introduced into the polymer compound nanofibers, negative charges that repel each other are carried between the polymer compound nanofiber chains by the sulfate group, the network structure of chains connected to each other by crosslinks is parted to a structure that can retain a large amount of water, and thus a transparent hydrogel with an increased degree of water swelling can be obtained.

Through Preparation Examples and Experimental Examples as described above, it can be confirmed that the three-dimensional structure of the hydrogel containing polymer nanofibers into which sulfate groups are introduced according to an embodiment of the present invention can be finely controlled by adjusting the diameter of nanofibers and the physical properties and degree of water swelling of the hydrogel are easily adjusted by adjusting the degree of crosslinking.

According to an embodiment of the present invention, it is possible to provide a hydrogel having a network structure, of which the degree of crosslinking and the degree of water swelling can be adjusted and the three-dimensional structure can be finely controlled by the crosslinking between polymer compound nanofibers and the introduction of a sulfate group.

According to an embodiment of the present invention, it is also possible to provide a hydrogel of which the three-dimensional structure can be finely controlled and thus which is highly utilizable as a biomaterial such as a three-dimensional cell culture scaffold and a bio-ink for 3D bioprinting technology.

According to an embodiment of the present invention, it is also possible to provide a hydrogel which is highly utilizable as an artificial extracellular matrix through a configuration that precisely mimics the extracellular matrix in vivo.

It should be understood that the effects of the present invention are not limited to the above-mentioned effects, and include all effects that can be inferred from the configuration of the invention described in the detailed description or claims of the present invention.

The above description of the present invention is for illustration, and those skilled in the art to which the present invention pertains will understand that it can be easily modified into other specific forms without changing the technical spirit or essential features of the present invention. Therefore, it should be understood that the embodiments described above are illustrative in all respects and not restrictive. For example, each component described as a single type may be implemented in a distributed manner, and similarly components described as being distributed may also be implemented in a combined form.

The scope of the present invention is indicated by the following claims, and all changes or modifications derived from the meaning and scope of the claims and their equivalents should be construed as being included in the scope of the present invention.

### REFERENCE SIGNS LIST

1: polymer compound nanofiber
2: crosslink
3: sulfate group

What is claimed is:

1. A hydrogel containing a polymer nanofiber into which a sulfate group is introduced, which comprises polymer compound nanofibers into which a sulfate group is introduced, wherein
the polymer compound nanofibers are connected to each other by a crosslink to form a network structure, and
gelling proceeds as water is filled in empty space of the network structure.

2. The hydrogel containing a polymer nanofiber into which a sulfate group is introduced according to claim 1, wherein the polymer compound includes at least any one selected from the group consisting of collagen, gelatin, elastin, fibrin, and fibronectin.

3. A method for preparing a hydrogel containing a polymer nanofiber into which a sulfate group is introduced, which comprises:
a polymer compound solution preparing step;
a crosslinking agent adding step of adding a crosslinking agent to the solution;
an electrospinning step of electrospinning the solution to form the polymer compound into polymer compound nanofibers;
a crosslink forming step of forming a crosslink between the polymer compound nanofibers;
a sulfate group introducing step of introducing a sulfate group into the polymer compound nanofibers; and
a hydrogel obtaining step of hydrating the resultant into which a sulfate group has been introduced to obtain a hydrogel.

4. The method for preparing a hydrogel containing a polymer nanofiber into which a sulfate group is introduced according to claim 3, wherein a diameter of the polymer compound nanofibers may be controlled by adjusting an electrospinning condition in the electrospinning step.

5. The method for preparing a hydrogel containing a polymer nanofiber into which a sulfate group is introduced according to claim 3, wherein the crosslinking agent contains a reducing sugar selected from the group consisting of glucose, fructose, maltose, lactose, and galactose.

6. The method for preparing a hydrogel containing a polymer nanofiber into which a sulfate group is introduced according to claim 3, wherein the crosslinking agent contains an organic acid having two or more carboxyl groups selected from the group consisting of sebacic acid, citric acid, and tartaric acid.

7. The method for preparing a hydrogel containing a polymer nanofiber into which a sulfate group is introduced according to claim 3, wherein the crosslinking agent contains at least any one selected from the group consisting of glutaraldehyde, genipin, and carbodiimide.

8. The method for preparing a hydrogel containing a polymer nanofiber into which a sulfate group is introduced according to claim 3, wherein the crosslinking agent may be added at a weight ratio of 0.06 to 0.2 with respect to the polymer compound.

9. The method for preparing a hydrogel containing a polymer nanofiber into which a sulfate group is introduced according to claim 3, wherein a sulfate group may be introduced into the polymer compound nanofibers by treating the polymer compound nanofibers with a sulfating agent in the sulfate group introducing step.

10. The method for preparing a hydrogel containing a polymer nanofiber into which a sulfate group is introduced according to claim 9, wherein the sulfating agent includes at least any one selected from the group consisting of concentrated sulfuric acid, chlorosulfonic acid, sulfur trioxide, dimethylformamide-sulfur trioxide complex, pyridine-sulfur trioxide complex, and oleum.

11. A three-dimensional artificial cell culture scaffold comprising the hydrogel containing a polymer nanofiber into which a sulfate group is introduced according to claim 1.

12. A bio-ink for 3D printing comprising the hydrogel containing a polymer nanofiber into which a sulfate group is introduced according to claim 1.
